(19) United States
(12) Patent Application Publication
(10) Pub. No.: US 2008/0139596 A1
(43) Pub. Date: Jun. 12, 2008

(54) Modulators of Hcv Replication

(76) Inventors: Raffaele De Francesco, Rome (IT); Petra Neddermann, Rome (IT)

Correspondence Address: MERCK AND CO., INC, P O BOX 2000, RAHWAY, NJ, 07065-0907, US

(21) Appl. No.: 11/661,350

(22) PCT Filed: Aug. 23, 2005
(86) PCT No.: PCT/EP05/09233

§ 371 Date: Nov. 23, 2007

(30) Foreign Application Priority Data
Aug. 27, 2004 (GB) 0419189.6

(51) Int. Cl. A61K 31/506; C12N 5/00; A61K 31/4545; A61P 31/12; C12Q 1/68; C12N 15/00; C12Q 1/48

(52) U.S. Cl. 514/275; 435/375; 514/318; 435/325; 435/6; 435/455; 435/15

(57) ABSTRACT

The present invention is directed to the use of certain 2,4,5-trisubstituted imidazole derivatives in modulating the replication of Hepatitis C virus RNA and/or virus production in cells.

The present invention is directed to the use of certain 2,4,5-trisubstituted imidazole derivatives in modulating the replication of Hepatitis C virus RNA and/or virus production in cells.

It is estimated that about 3% of the world's population are infected with the Hepatitis C virus (HCV) (Wasley, et al., 2000, Semin. Liver Dis. 20, 1-16). Exposure to HCV results in an overt acute disease in a small percentage of cases, while in most instances the virus establishes a chronic infection causing liver inflammation and slowly progresses into liver failure and cirrhosis (Iwarson, 1994, FEMS Microbiol. Rev. 14, 201-204). In addition, epidemiological surveys indicate an important role of HCV in the pathogenesis of hepatocellular carcinoma (Kew, 1994, FEMS Microbiol. Rev. 14, 211-220, Alter, 1995. Blood 85, 1681-1695).

Investigating the effects of HCV and antiviral compounds is complicated by the absence of a way to reproduce infection in laboratory small animal models as well as in cultivated cells. HCV infects human and chimpanzees, but does not infect small animals such as mice and rats. Similarly, HCV does not efficiently propagate in any cultivated cells or tissues.

Lohmann et al., Science 285, 110-113, 1999 disclose a HCV cell culture system where the viral RNA self-replicates in the transfected cells efficiently, and illustrate the ability of a biscistronic HCV subgenomic replicon to replicate in a hepatoma cell line. An HCV replicon is an RNA molecule able to autonomously replicate in a cultured cell and produce detectable levels of one or more HCV proteins.

HCV replicons can thus be used to produce a cell culture providing detectable levels of HCV RNA and HCV protein. In order to replicate efficiently, however, these replicons require the presence of adaptive mutations (see for example, Lohmann et al., J Virol 77, 3007-3019, 2003).

Adaptive mutations are mutations in HCV RNA that enhance the ability of an HCV replicon to be maintained and expressed in a host cells. Examples of adaptive mutations can be found in U.S. Pat. No. 6,630,343 B1; WO2002059321 A2; WO0189364 A2; Bartenschlager et al., Antiviral Res. 60, 91-102, 2003, and references therein.

Many adaptive mutations map in the viral protein NS5A, in some cases affecting its phosphorylation status. HCV NS5A is a 446-amino acid phosphoprotein, which is phosphorylated on serine/threonine residues and that exists in two distinct species, termed p56 (phosphorylated) and p58 (hyperphosphorylated). Adaptive mutations can result in a significant reduction of the formation of p58, i.e. the hyperphosphorylated form of NS5A.

It has now surprisingly been found that pharmacological agents can prevent NS5A hyperphosphorylation, and therefore can be used to support replication of HCV RNA in cell culture without the need to introduce adaptive mutations. Such cell culture system is a better mimic of in vivo replication and is useful in supporting replication of naturally occurring HCV sequences and assisting the establishment of HCV viral infection assays in cultured cells and test animals.

It has been found that pharmacological agents that prevent NS5A hyperphosphorylation can modulate the replication of HCV RNA also to the extent that HCV RNA replication is inhibited. Inhibitors of NS5A hyperphosphorylation may thus have therapeutic applications to treat individuals infected with HCV.

Thus, in one aspect, the present invention provides the use of an agent to inhibit the formation of hyperphosphorylated NS5A in a cell, a tissue or an organism.

Preferably, the agent is:
N-methyl-4-{2-piperidin-4-yl-4-[3-(trifluoromethyl)phenyl]-1H-imidazol-5-yl}pyrimid-2-amine (1),
4-[5-(4-fluorophenyl)-2-(1-methylpiperidin-4-yl)-1H-imidazol-4-yl]pyridine (2), or
4-[5-(4-fluorophenyl)-4-pyridin-4-yl-1H-imidazol-2-yl]piperidine (3),
   or a suitable salt thereof.

In a further aspect, the present invention provides the use of an agent to modulate the replication of HCV RNA and/or viral production of HCV in a cell, a tissue or an organism.

Preferably, the agent is compound (1), (2) or (3), or a suitable salt thereof.

In a further aspect, the present invention provides a method for modulating the replication of HCV RNA and/or viral production of HCV in a cell, a tissue or an organism comprising administering to the cell, the tissue or the organism an agent which inhibits the formation of hyperphosphorylated NS5A.

Preferably, the agent which inhibits the formation of hyperphosphorylated NS5A is compound (1), (2) or (3), or a suitable salt thereof.

The skilled addressee will appreciate that references herein to “modulation” and the like of replication of HCV RNA or viral production of HCV is intended to include the inhibition and enhancement of HCV RNA replication or HCV production.

Thus, in one embodiment, there is provided the use of compound (1), (2) or (3), or a suitable salt thereof, to enhance HCV RNA replication and/or viral production of HCV in a cell.

In a further embodiment, there is provided a method of enhancing HCV RNA replication and/or viral production of HCV in a cultured cell by treating the cell with compound (1), (2) or (3) or a suitable salt thereof.

In a further aspect, the present invention provides a cell culture obtainable by treatment with compound (1), (2) or (3) or a suitable salt thereof.

The skilled addressee will appreciate that references herein to HCV RNA are intended to include sub-genomic replicons and full length HCV RNAs. Full length HCV RNA can be introduced into a cell by transfection of HCV RNA or by inoculating the cell with HCV virus obtained from infected individuals or produced in cell culture.

Enhancing HCV RNA replication in a cell with the compounds of the present invention brings about at least one of the following: an increase in maintenance of HCV RNA replication, an increase in the rate of HCV RNA replication, an increase in HCV RNA expression, an increase in HCV protein expression, and an increase in virus production.

Enhancing replication and expression of HCV RNA in a cell culture system using the compounds of the present invention has a variety of different uses, including being used to study HCV replication and expression, to study HCV and host cell interactions, to produce HCV RNA, to produce HCV proteins, to assist in establishing HCV viral infection in cell culture and to provide a system for measuring the ability of a compound to modulate one or more HCV activities.

In a further aspect, the present invention provides a method of screening a compound for its effect on HCV replication which comprises administration of the compound to a HCV cell culture that has been treated with compound (1), (2) or (3) or a suitable salt thereof.

The compounds described in this invention can be used to produce a cell culture providing detectable levels of HCV RNA and HCV protein in the absence of adaptive mutations that are specific for given cell culture conditions, cell lines or HCV viral isolates. Moreover, the compounds described in the present invention can be exploited to enable replication, in cultivated cells, of HCV RNA with naturally occurring sequences representing different isolates and genotypes.

Thus, in a further aspect, the present invention provides the use of compound (1), (2) or (3) or a suitable salt thereof in the production of a cell culture which has detectable levels of HCV RNA and HCV protein in the absence of adaptive mutations in the HCV RNA.

In a further aspect, the present invention provides a method of producing a cell culture which has detectable levels of HCV RNA in the absence of adaptive mutations in the HCV RNA by:
a) contacting a cell in tissue culture with HCV RNA or HCV virus not carrying adaptive mutations,
b) treating the cell with compound (1), (2) or (3) or a suitable salt thereof,
c) evaluating the treated cell for HCV RNA replication.

In a further aspect, the present invention provides a method for producing a cell culture which has detectable levels of HCV protein in the absence of adaptive mutations in the HCV RNA by:
a) contacting a cell in tissue culture with HCV RNA or HCV virus not carrying adaptive mutations,
b) treating the cell with compound (1), (2) or (3) or a suitable salt thereof,
c) evaluating the treated cell for HCV protein expression.

In a further aspect, the present invention provides a method of producing a cell culture which has detectable levels of virus production in the absence of adaptive mutations by:
a) contacting a cell in tissue culture with HCV RNA or HCV virus not carrying adaptive mutations,
b) treating the cell with compound (1), (2) or (3) or a suitable salt thereof,
c) evaluating the amount of viral particles secreted in the cell medium.

The compounds described in this invention can also be used in combination with selected adaptive mutations present in HCV variants in order to assist the establishment of detectable HCV RNA replication and HCV protein expression in cultivated cells.

Thus, in a further aspect, the present invention provides the use of compound (1), (2) or (3) or a suitable salt thereof in the production of a cell culture which has detectable levels of HCV RNA and HCV protein in the presence of selected adaptive mutations in those cells.

In a further aspect, the present invention provides a method of producing a cell culture which has detectable levels of HCV RNA in the presence of selected adaptive mutations in those cells by:
a) contacting a cell in tissue culture with HCV RNA or HCV virus carrying selected adaptive mutations,
b) treating the cell with compound (1), (2) or (3) or a suitable salt thereof,
c) evaluating the treated cell for HCV RNA replication.

In a further aspect, the present invention provides a method of producing a cell culture which has detectable levels of HCV protein in the presence of selected adaptive mutations in those cells by:
a) contacting a cell in tissue culture with HCV RNA or HCV virus carrying adaptive mutations,
b) treating the cell with compound (1), (2) or (3) or a suitable salt thereof,
c) evaluating the treated cell for HCV protein expression.

In a further aspect, the present invention provides a method of producing a cell culture which has detectable levels of virus production in the presence of adaptive mutations by:
a) contacting a cell in tissue culture with HCV RNA or HCV virus carrying adaptive mutations,
b) treating the cell with compound (1), (2) or (3) or a suitable salt thereof,
c) evaluating the amount of viral particles secreted in the cell medium.

Cell systems suitable for use in the present invention include, but are not restricted to, primary human cells, for example hepatocytes, T-cells, B-cells and foreskin fibroblasts, as well as continuous human cell lines, for example HuH7, HepG2, HUT78, HPB-MA, MT-2, MT-2C, and other HTLV-1 and HTLVII infected T-cell lines, Namalawa, Daudi, EBV-transformed LCLs. In addition, cell lines of other species, especially those that are permissive for replication of flaviviruses or pestiviruses, for example SW-13, Vero, BHK-21, COS, PK-15, MBCK, etc., can be used.

Preferred cell systems are hepatoma cell lines such as Huh-7, Hep3B and HepG2.

The skilled person will appreciate that the uses and methods described herein to modulate HCV RNA replication and/or HCV virus production in cell cultures can be adapted to modulate HCV RNA replication, HCV virus infection and/or HCV virus production in test animals.

Test animals suitable for use in the present invention include mammals such as rodents. Preferred test animals are rodents such as rats and mice.

The presence of replicating HCV RNA can be evaluated by conventional methods such as, for example, RT-PCR, quantitative RT-PCR, Northern blotting, or by measuring the activity of an HCV protein or protein encoded by reporter gene engineered into the HCV RNA.

HCV protein expression can be evaluated by conventional methods such as, for example, ELISA assays, Western Immunoblots, or radioactive protein labeling followed by immunoprecipitation assays.

The presence of HCV viral particles secreted in the cell medium can be evaluated by conventional methods, such as, for example, real-time reverse transcription PCR amplification (TaqMan), b-DNA, or by utilizing the cell medium to infect naïve cells or laboratory animals.

The compounds described in this invention can also be used in order to identify the cellular kinase(s) responsible for the hyperphosphorylation of HCV NS5A.

Thus, in a further aspect, the present invention provides the use of compound (1), (2) or (3) or a suitable salt thereof in the identification of cellular kinase(s) responsible for the hyperphosphorylation of HCV NS5A.

In a further aspect, the present invention provides a method of identifying cellular kinase(s) responsible for the hyperphosphorylation of HCV NS5A by:
a) covalently binding compound (1), (2) or (3) or a suitable salt thereof to a chromatography matrix,
b) using the chromatography matrix to purify kinase(s) from cellular protein extracts,
c) eluting the kinase(s) from the affinity matrix,
d) identifying the eluted kinase(s).

The level of NS5A hyperphosphorylation needs to be tightly regulated during the viral replication. It has been found that varying the concentration of compounds (1), (2) or (3) can modulate the replication of HCV RNA to the extent that HCV RNA replication is inhibited. Inhibitors of NS5A hyperphosphorylation may thus have therapeutic applications to treat HCV patients.

Thus, in a further aspect, the present invention provides the use of compound (1), (2) or (3) or a pharmaceutically acceptable salt thereof in the manufacture of a medicament for the treatment of HCV infection.

In another aspect of the invention, there is provided a method of inhibiting replication of HCV RNA and/or of treating or preventing an illness due to hepatitis C virus, the method involving administering to a human or animal (preferably mammalian) subject suffering from the condition a therapeutically or prophylactically effective amount of the pharmaceutical composition described above or of compound (1), (2) or (3) as defined above, or a pharmaceutically acceptable salt thereof. “Effective amount” means an amount sufficient to cause a benefit to the subject or at least to cause a change in the subject's condition.

In a further embodiment of the present invention, there is provided the use of compound (1), (2) or (3), or a pharmaceutically acceptable salt thereof, for the manufacture of a medicament for the treatment or prevention of infection by hepatitis C virus, in combination with one or more other agents for the treatment of viral infections such as an antiviral agent, and/or an immunomodulatory agent such as α, β- or γ-interferon, particularly α-interferon. Suitable antiviral agents include ribavirin and inhibitors of hepatitis C virus (HCV) replicative enzymes, such as inhibitors of metalloprotease (NS2-3), serine protease (NS3), helicase (NS3) and RNA-dependent RNA polymerase (NS5B).

Suitable pharmaceutically acceptable salts of the compounds of this invention include acid addition salts which may, for example, be formed by mixing a solution of the compound according to the invention with a solution of a pharmaceutically acceptable acid such as hydrochloric acid, fumaric acid, p-toluenesulfonic acid, maleic acid, succinic acid, acetic acid, citric acid, tartaric acid, carbonic acid, phosphoric acid or sulfuric acid. Salts of amine groups may also comprise quaternary ammonium salts in which the amino nitrogen atom carries a suitable organic group such as an alkyl, alkenyl, alkynyl or aralkyl moiety.

Suitable salts of the compounds of the present invention include, not only the pharmaceutically acceptable salts thereof as hereinbefore described, but also any common salts or quaternary ammonium salts formed, e.g., from inorganic and organic acids. Suitable salts include those derived from inorganic acids such as hydrochloric, hydrobromic, sulfuric, sulfamic, phosphoric, nitric and the like: and the salts prepared from organic acids such as acetic, propionic, succinic, glycolic, stearic, malic, tartaric, citric, ascorbic, mapoic, sulfanilic, 2-acetoxybenzoic, fumaric, toluenesulfonic, methane-sulfonic, ethane disulfonic, oxalic, isethionic, trifluoroacetic and the like. The salts are generally prepared by reacting the free base or acid with stoichiometric amounts or with an excess of the desired salt-forming inorganic or organic acid or base in a suitable solvent or solvent combination.

The present invention includes within its scope prodrugs of compounds (1), (2) or (3) above. In general, such prodrugs will be functional derivatives of compounds (1), (2) or (3) which are readily convertible in vivo into the required compounds (1), (2) or (3). Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulfate ester, or reduction or oxidation of a susceptible functionality.

Compound (1) is disclosed in published International patent application WO 97/47618 (Merck & Co., Inc.), and compounds (2) and (3) are disclosed in published International patent application WO 97/36587 (Merck & Co., Inc.). The syntheses of compounds (1) and (2) are shown in the following schemes:

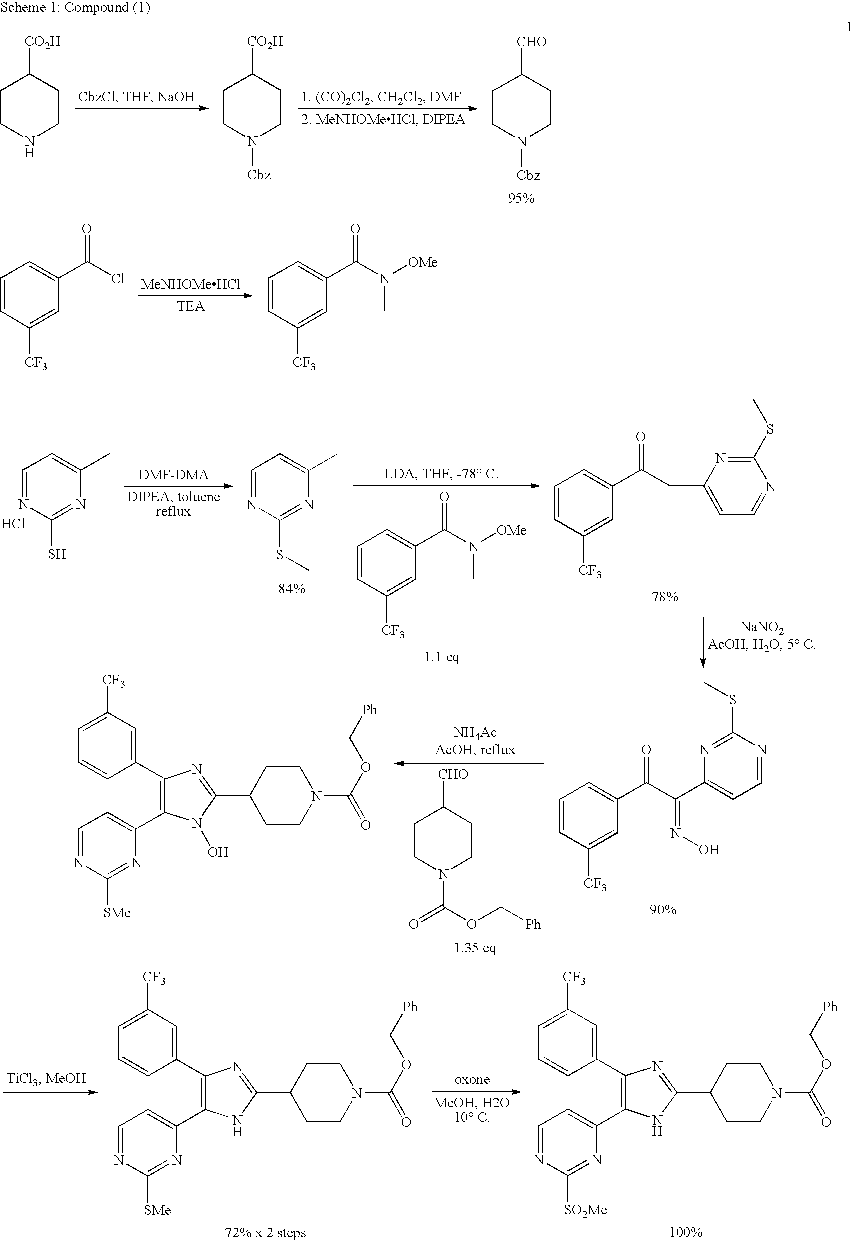

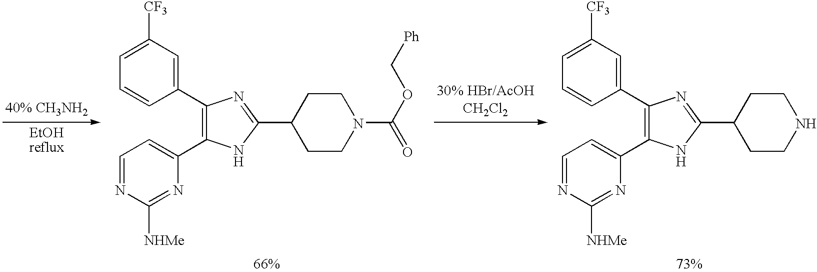

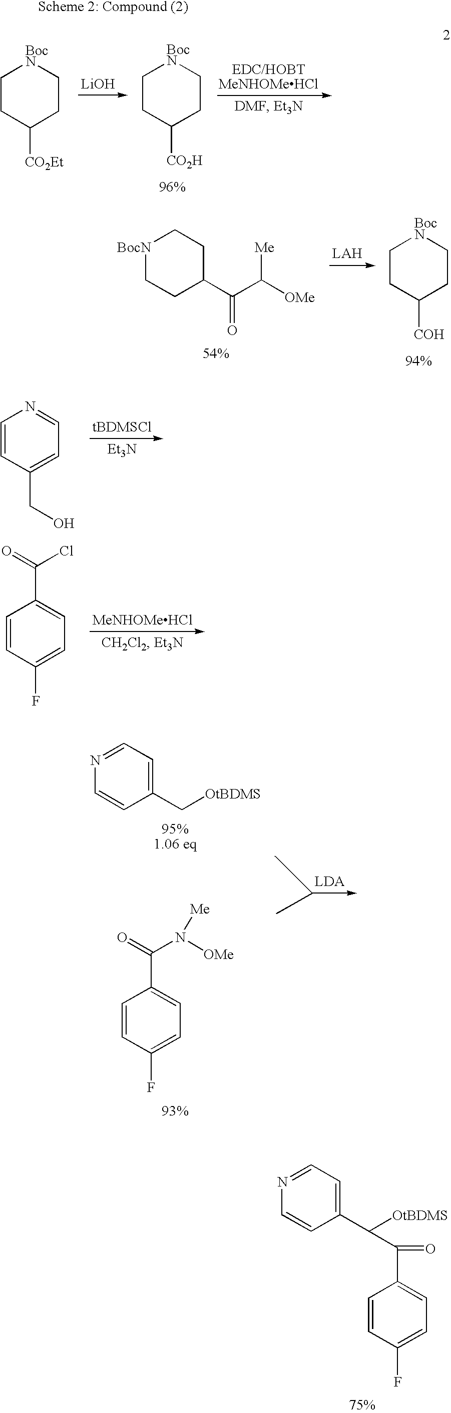

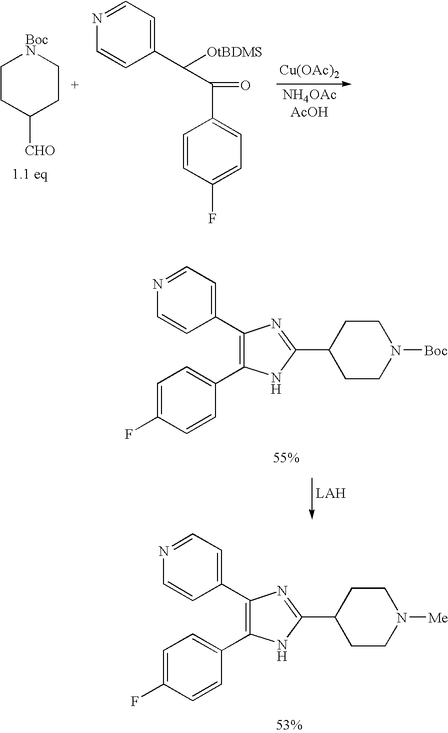

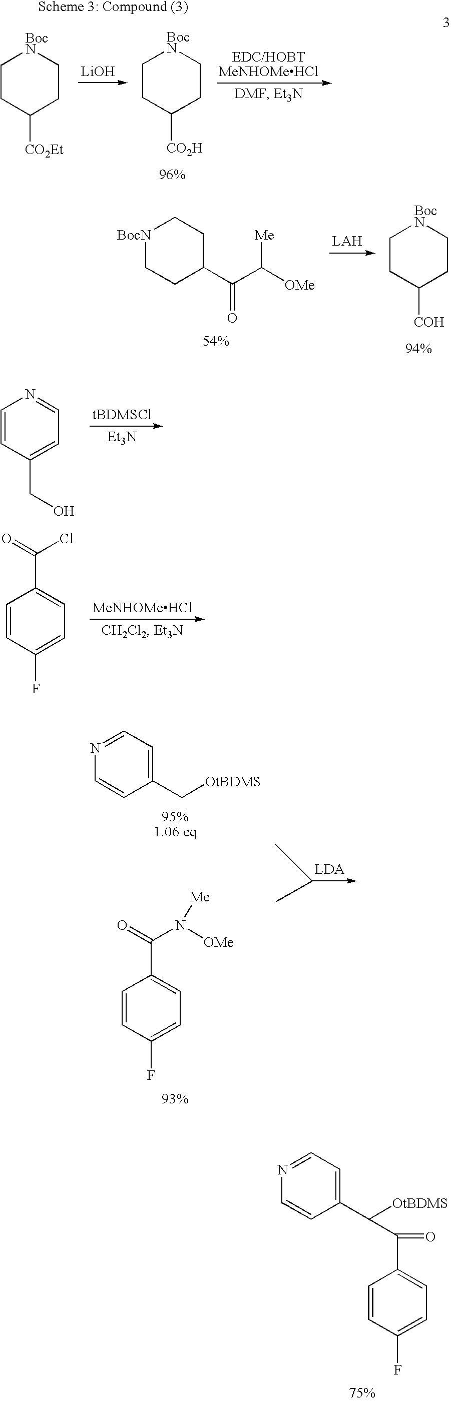

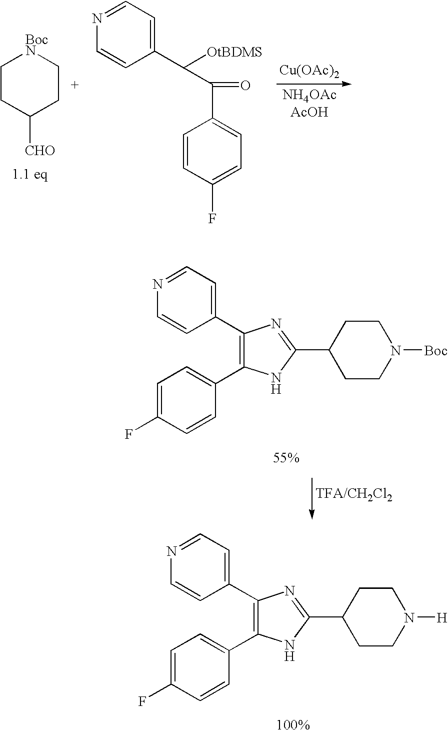

The abbreviations used in these schemes are as follows:
Ac=acetyl; tBDMS=tert Butyldimethylsilyl; Boc=tert Butyloxycarbonyl; Cbz or Z=benzyloxycarbonyl; DIPEA=N,N-Diisopropylethylamine; DMF=N,N-dimethylformamide; EDC=1-ethyl-(3-dimethyl amino propyl) carbodiimide hydrochloride; HOBT=1-Hydroxybenzotriazole; LAH=Lithium aluminium hydride; LDA=Lithium diisopropylamide; TEA=Triethylamine

The invention is illustrated by the accompanying Figures.

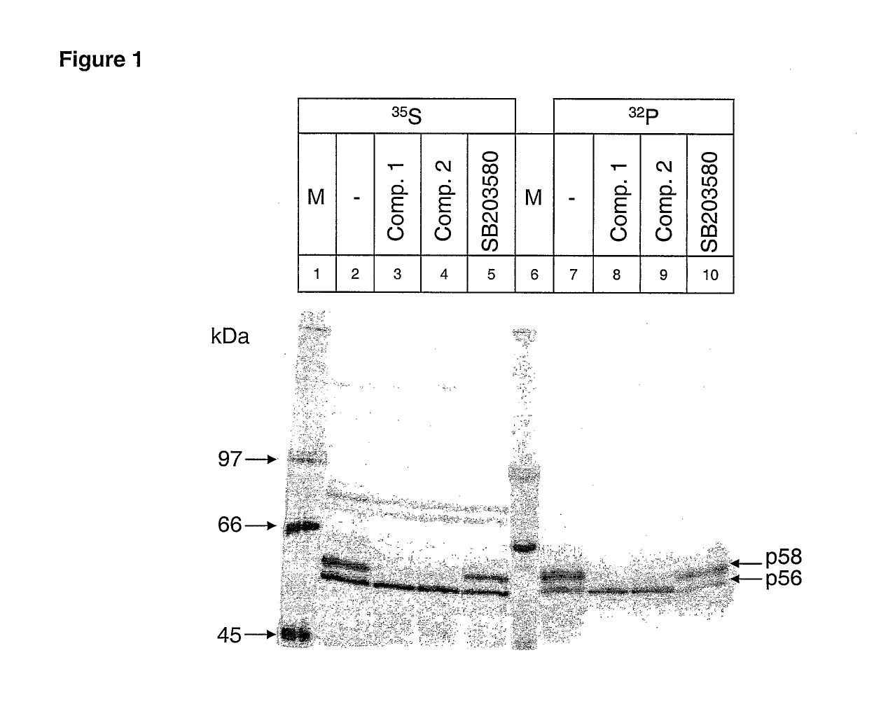

FIG. 1—Inhibition of NS5A hyperphosphorylation in cell culture

The presence of hyperphosphorylated NS5A (p58) was evaluated in Huh7-HB68 cells. Proteins were labeled either with ³⁵S-methionine (lanes 2-5) or with ³²P-orthophosphate (lanes 7-10) in the presence of DMSO (lanes 2 and 7) or with 5 μM of Compound (1) (lanes 3 and 8), Compound (2) (lanes 4 and 9) or SB203580 (lanes 5 and 10). After the radiolabelling, protein extract was prepared, NS5A was immunoprecipitated and proteins were loaded on a 7.5% SDS-PAGE and autoradiographed. The sizes of molecular weight marker proteins are indicated in lanes 1 and 6.

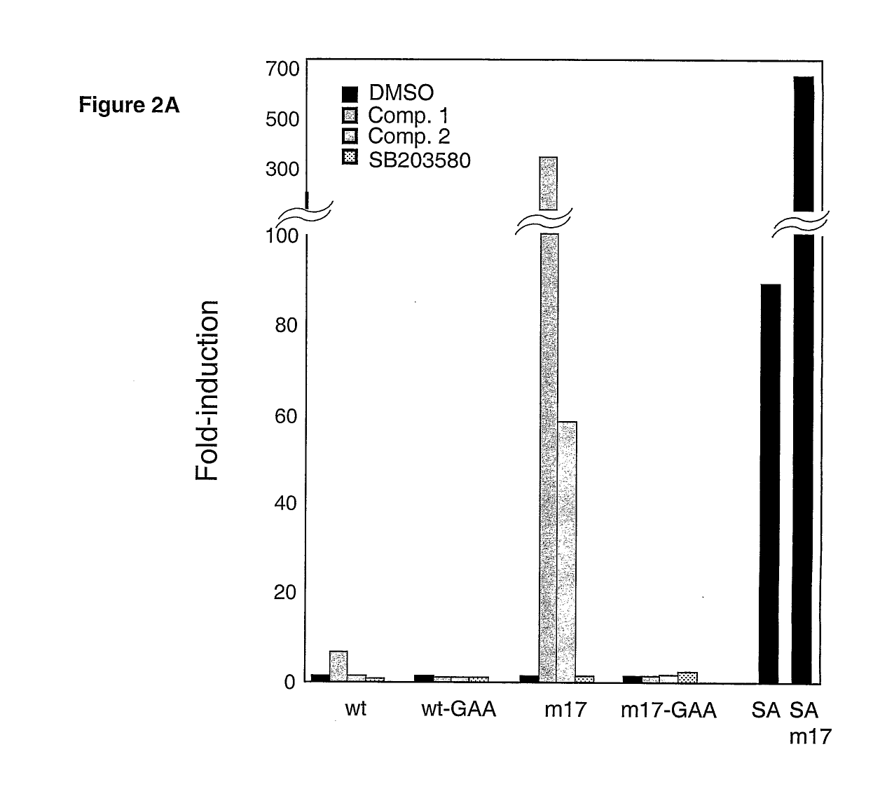

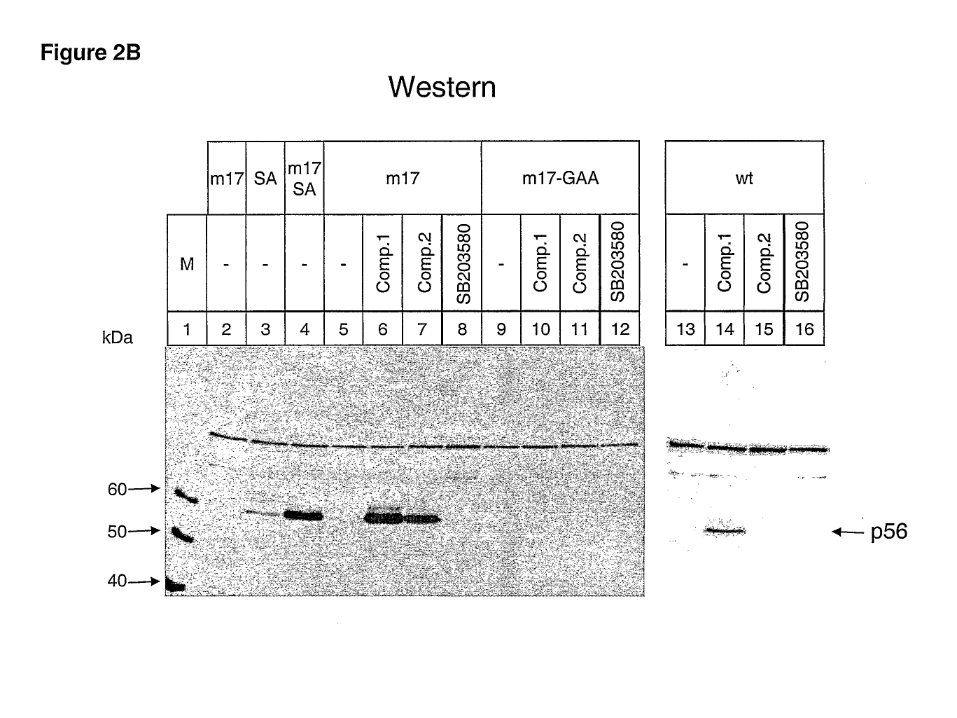

FIG. 2—Detection of HCV-RNA and HCV-specific proteins after treatment of the cells with compounds described in the invention

RNA was transcribed from the plasmids wt, wt-GAA, m17, SA, m17/SA and m17-GAA and electroporated into 10A-IFN cells. Cells were incubated for four days without or with 8 μM of Compound (1), Compound (2) or SB203580.

(A) HCV-specific RNA analysis using quantitative RT-PCR. Total cellular RNA was extracted and HCV-specific RNA was quantified as described in Materials and Methods. On the Y-axis is shown the fold-induction with respect to the DMSO control (black bar), Compound (1) (dark grey bar), Compound (2) (light grey bar) or SB203580 (dotted bar). Replicon RNA is indicated at the bottom of the figure.

(B) Western Blot of total protein extract. Cell extract was prepared and 50 μg of protein were loaded onto SDS-PAGE for each lane. Specific anti-NS5A antibody was used as primary antibody and a Peroxidase-conjugated antibody (Pierce) was used as secondary antibody. The Western Blot was developed using the SuperSignal West Pico Chemiluminescent Substrate (Pierce).

### NS5A HYPERPHOSPHORYLATION ASSAY

Inhibition of NS5A hyperphosphorylation in intact cells was measured by determining the amount of hyperphosphorylated NS5A (p58) in cells expressing HCV NS5A in the context of a polyprotein comprising at least NS3, NS4A, NS4B, and NS5A (Neddermann et al., J Virol 73, 9984-9991, 1999). Suitable cells are, for example, cells stably expressing an HCV replicon with adaptive mutations that do not affect NS5A hyperphosphorylation, such as HBI10 or HB68. HBI10 and HB68 are Huh-7 derived-cell lines described in WO2002/059321.

Hyperphosphorylated NS5A was detected as a protein that i) migrates with an apparent molecular weight of about 58 kDa in SDS PAGE, and ii) is immunoreactive with anti-NS5A antibodies. Thus, the amount of hyperphosphorylated NS5A was detected by immunoprecipitation of radioactively labeled proteins.

### Materials

### Cell Culture

HBI10 or HB68 were cultured in Dulbecco's modified Eagle medium (DMEM) supplemented with 10% fetal bovine serum (FBS) in the presence of 0.8 mg/ml of G418 (Geneticin; Gibco/BRL). For starvation prior to radioactive protein labeling, minimal essential medium without methionine (Gibco/BRL) was used. For protein labeling, 100 μCi/ml of ³⁵S-labelled methionine (Promix, Amersham, Cat. No. SJQ0079, 1000 Ci/mmole) was added to the cells.

### Cell Lysis Buffer

25 mM sodium phosphate pH 7.5, 20% glycerol, 1% Triton X-100, 150 mM NaCl, 1 mM EDTA, 2 mM dithiothreitol (DTT), 2 mM phenylmethylsulfonyl fluoride (PMSF).

### Immunoprecipitation Buffer

20 mM Tris-HCl pH 8, 150 mM NaCl, 1% Triton X-100

### NDET Buffer

10 mM Tris-HCl pH 7.50, 4% sodium deoxycholate, 0.5% Triton X-100, 10 mM EDTA

### Protein A-Sepharose Resin

Protein A-Sepharose resin was obtained from Amersham Biosciences

### Antibodies

NS5A-specific antisera were obtained as described in Tomei et al., J. Virol. 67, 4017-4026, 1993.

### Method

1. HBI10 or HB68 cells were grown to 80% confluency in 6-well plates.
2. Compound to be tested, dissolved in DMSO at 100× concentration, was added to each well.
3. One hour later, the medium was removed and replaced with Minimal Essential Medium without methionine. This step is omitted in the case of 32P-orthophosphate labeling.
4. Compound to be tested, dissolved in DMSO at 100× concentration, was freshly added to each well.
5. One hour later 100 μCi of ³⁵S-labelled methionine per ml of Minimal Essential Medium without methionine was added to each well for 35S-metabolic labeling together with the compound to be tested, dissolved in DMSO at 100× concentration. In the case of 32P-labelling, cells were washed once with Dulbecco's modified Eagle's medium without phosphate (ICN) and labeled for 4 hours in the same medium containing 500 μCi/ml of [32P]-orthophosphate (285.5 Ci/mg, NEN) together with the compound to be tested, dissolved in DMSO at 100× concentration.
6. After 4 hours, cells were harvested from each well and individual cell extracts were prepared in 100 μl of cell lysis buffer.
7. 50 μl of each extract were then heated at 95° C. for 4 min after the addition of 2% sodiumdodecyl sulfate (SDS) and 10 mM DTT.
8. Aliquots of antibody coated protein A-Sepharose for immunoprecipitation of the extracts were prepared by mixing 5 μl of HCV NS5A-specific antisera and 50 μl of protein A-Sepharose 50% suspension in 300 μl of immunoprecipitation buffer and incubating under gentle stirring for 1 h at 4° C.
9. The antibody-coated protein A-Sepharose thus obtained was then washed twice with 300 μl of immunoprecipitation buffer and resuspended in 500 μl of the same buffer.
10. The radiolabelled protein extracts obtained at step 7 were added to the suspension and the mixtures incubated under gentle stirring for 1 h at 4° C.
11. The immunoprecipitate was collected and resuspended and the mixture layered on 0.7. ml of 0.5×NDET buffer containing 30% sucrose and pelleted by centrifugation for 5 min at 5,000×g.
12. The immunoprecipitate was then washed once with 500 μl of NDET and once with 500 μl of PBS and
13. Protein was detached from the PAS-resin by boiling in SDS sample buffer and loaded on a 7.5% SDS-PAGE for electrophoresis. When the dye front reached the bottom of the gel, the gel was fixed, soaked in Amplify (Amersham Bioscience) for 30 minutes, dried, and autoradiographed on an X-ray film or a Phosphoimager (Storm 820, Amersham Pharmacia Biotech) in order to evaluate the amount of hyperphosphorylated NS5A. The intensities of the bands corresponding to hyperphosphorylated NS5A and non-hyperhosphorylated NS5A were compared to determine the percent inhibition of NS5A hyperphosphorylation.

### Agents that inhibit the formation of hyperphosphorylated NS5A were tested for inhibitory activity in the assay described above and the compounds were generally be found to have IC₅₀ values in the range from about 0.001 μM to about 50 μM.

### Methods to Detect HCV Replication

To determine the biological consequences of inhibition of NS5A hyperphosphorylation, the effect of the compounds of the present invention were tested on HCV RNA replication in cell culture.

Methods for detecting HCV RNA replication include those measuring the production or activity of HCV RNA, production or activity of viral proteins or production of viral particles. Measuring includes qualitative and quantitative analysis.

Techniques suitable for measuring RNA production include those detecting the presence or activity of RNA. The presence of RNA can be detected using, for example, complementary hybridization probes or quantitative RT-PCR or Northern blotting. Techniques for measuring hybridization between complementary nucleic acid and quantitative PCR are well known in the art (see for example, Ausubel, Current Protocols in Molecular Biology, John Wiley, 1987-1998, Sambrook, et al., Molecular Cloning, A Laboratory Manual, 2^{nd} Edition, Cold Spring Harbor Laboratory Press, 1989, and U.S. Pat. No. 5,731,148).

Techniques for measuring protein production include those detecting the presence or activity of a produced protein. The presence of a particular protein can be determined by, for example, immunological techniques such as ELISA assays, Western Immunoblots, or radioactive protein labeling followed by immunoprecipitation assays. Protein activity can be measured based on the activity of an HCV protein or a reporter protein sequence.

Techniques for measuring HCV protein activity vary depending upon the protein that is measured. Techniques for measuring the activity of different non-structural proteins such as NS2/3, NS3, and NS5B, are well known in the art (see, for example, references hereinbefore provided).

Assays measuring HCV RNA replication also include those detecting virion production from a replicon that produces a virion. The presence of HCV viral particles secreted in the cell medium can be evaluated by conventional methods, such as, for example, real-time reverse transcription PCR amplification (TaqMan), b-DNA, or by utilizing the cell medium to infect naïve cells or laboratory animals. Assays measuring HCV RNA replication also include those detecting a cytopathic effect from a replicon producing proteins exerting such an effect. Cytopathic effects can be detected by assays suitable to measure cell viability.

A reporter sequence can be used to detect HCV RNA replication or protein expression. Preferred reporter proteins are enzymatic proteins whose presence can be detected by measuring product produced by the protein. Examples of reporter proteins include luciferase, beta-lactamase, secretory alkaline phosphatase, beta-glucuronidase, green fluorescent protein and its derivatives. In addition, a reporter nucleic acid sequence can be used to provide a reference sequence that can be targeted by a complementary nucleic acid. Hybridization of the complementary nucleic acid to its target can be determined using standard techniques.

Assays measuring HCV RNA replication can be used to evaluate the ability of a compound to modulate HCV RNA replication. Such assays can be carried out by providing one or more test compounds to a cell expressing an HCV RNA and measuring the effect of the compound on RNA replication.

### EXAMPLES

Examples are provided below to further illustrate different features of the present invention. The examples also illustrate useful methodology for practicing the invention. These examples do not limit the claimed invention.

### Example 1

### Materials and Techniques

This example illustrates the techniques employed for evaluating the biological effects of the compounds of the present invention

### Cells and Cell Culture

HBI10A, HB68 and 10AIFN were derived from Huh-7 cells as described in WO2002059321 A2; Mottola et al., Virology 293, 31-43, 2002; and Trozzi et al., J Virol 77, 3669-3679, 2003.

Cells were cultured in Dulbecco's modified Eagle medium (DMEM) supplemented with 10% fetal bovine serum (FBS) and in the case of HBI10A and HB68 in the presence of 0.8 mg/ml of G418 (Geneticin; Gibco/BRL). For routine work, cells were passed 1 to 5 twice a week using 1× trypsin/EDTA (Gibco, BRL).

### Nucleic Acids and Construction of Recombinant Plasmids

Manipulation of nucleic acids was done according to standard protocols (Sambrook, et al., 1989. Molecular Cloning: A Laboratory Manual, 2^{nd} ed. Cold Spring Harbor Laboratory, Cold Spring Harbor, N.Y.) Plasmid DNA was prepared from ON culture in LB broth using Qiagen 500 columns according to manufacturer instructions.

Plasmids containing desired mutations were constructed by restriction digestion using restriction sites flanking the mutations or by PCR amplification of the area of interest, using synthetic oligonucleotides with the appropriate sequence. Site directed mutagenesis was carried out by inserting the mutations in the PCR primers. PCR amplification was performed using high fidelity thermostable polymerases or mixtures of polymerases containing a proofreading enzyme (Barnes, et al., 1994. Proc. Natl. Acad. Sci. 91, 2216-2220.) All plasmids were verified by restriction mapping and sequencing.

pHCVneo17.wt is described in Trozzi et al., J Virol 77, 3669-3679, 2003. It contains the cDNA for an HCV bicistronic replicon identical to replicon I₃₇₇neo/NS3-3′/wt described by Bartenschlager (SEQ. ID. NO. 3) (Lohmann et al., 1999. Science 285, 110-113, EMBL-genbank No. AJ242652). The plasmid comprises the following elements: 5′ untranslated region of HCV comprising the HCV-IRES and part of the core (nt1-377); neomycin phosphotransferase coding sequence; and EMCV IRES; HCV coding sequences from NS3 to NS5B; 3′ UTR of HCV. pHCVNeo17.C is a variant of pHCVneo17.wt as described in Trozzi et al., supra. The other plasmids are identical to pHCVNeo17.wt but contain the following mutations: (i) SA, S2204A in NS5A; (ii) S1, S2204I in NS5A; (iii) AT, A2199T in NS5A; (iv) m17/SA, S2204A in NS5A and E1202G in NS3; (v) m17-GAA, E1202G in NS3 and D2737A/D2738A in NS5B; (vi) wt-GAA, D2737A/D2738A in NS5B. For the Bla-reporter HCV replication assay the neomycin phosphotransferase (neo) gene of plasmid pHCVneo17.wt and pHCVneo17.SA was replaced by the β-lactamase (bla) gene as described in WO2003089672 A1 and in Murray et al., J Virol 77, 2928-2935, 2003, resulting in plasmid wt-BLA and SA-BLA, respectively.

### RNA Transfection

Plasmids were digested with the ScaI endonuclease (New England Biolabs) and transcribed in vitro with the T7 Megascript kit (Ambion). Transcription mixtures were treated with DNase I (0.1 U/ml) for 30 minutes at 37° C. to completely remove template DNA, extracted according to the procedure of Chomczynski (Chomczynski et al., 1987. Anal. Biochem. 162, 156-159), and resuspended with RNase-free phosphate buffered saline (rfPBS, Sambrook et al., 1989. Molecular Cloning: A Laboratory Manual, 2^{nd} ed. Cold Spring Harbor Laboratory, Cold Spring Harbor, N.Y.).

RNA transfection was performed as described by Liljestrom et al., 1991. J. Virol. 6, 4107-4113, with minor modifications. Subconfluent, actively growing cells were detached from the tissue culture container using trypsin/EDTA. Trypsin was neutralised by addition of 3 to 10 volumes of DMEM/10% FCS and cells were centrifuged for 5 minutes at 1200 rpm in a Haereus table top centrifuge at 4° C. Cells were resuspended with ice cold rfPBS by gentle pipetting, counted with a haemocitometer, and centrifuged as above. rfPBS wash was repeated once and cells were resuspended at a concentration of 1-2×10⁷ cell/ml in rfPBS. Aliquots of cell suspension were mixed with RNA in sterile eppendorf tubes. The RNA/cell mixture was immediately transferred into the electroporation cuvette (precooled on ice) and pulsed twice with a gene pulser apparatus equipped with pulse controller (Biorad). Depending on the experiment, 0.1, 0.2 or 0.4 cm electrode gap cuvettes were used, and settings adjusted (see Table below).

TABLE

| Cuvette | Volume | Voltage | Capacitance | Resistance | RRNA |
|---|---|---|---|---|---|
| gap (cm) | (μl) | (Volts) | (μFa) | (ohm) | (μg) |
| 0.1 | 70 | 200 | 25 | infinite | 1-10 |
| 0.2 | 200 | 400 | 25 | infinite | 5-20 |
| 0.4 | 800 | 800 | 25 | infinite | 15-100 |

After the electric shock, cells were left at room temperature for 1-10 minutes (essentially the time required to electroporate all samples) and subsequently diluted with at least 20 volumes of DMEM/10% FCS and plated as required for the experiment. Survival and transfection efficiency were monitored by measuring the neutral red uptake of cell cultured for various days in the absence or in the presence of neomycin sulfate (G418). With these parameters, survival of Huh-7 cells was usually 40-60% and transfection efficiency ranged between 40% and 100%.

### Real-Time Reverse Transcription PCR Amplification (TaqMan)

Replicon RNA was extracted from selected clones either using the Qiagen RNAeasy minikit following manufacturer instructions or as described by Chomczynski et al., 1987. Anal. Biochem. 162, 156-159. TaqMan analysis was typically performed using 10 ng of RNA in a reaction mix (TaqMan Gold RT-PCR kit, Perkin Elmer Biosystems) either with HCV specific oligos/probe (as disclosed in published International application WO02/059321) or with human beta-actin specific oligos/probe (Pre-Developed TaqMan Assay Reagents, Endogenous Control Human beta-actin, Part Number 4310881E, Applied Biosystems). PCR was performed using a Perkin Elmer ABI PRISM 7700 under the following conditions: 30 minutes at 48° C. (the RT step), 10 minutes at 95° C. and 40 cycles: 15 seconds at 95° C. and 1 minute at 60° C. Quantitative calculations were obtained using the Comparative C_{T} Method (described in User Bulletin #2, ABI PRISM 7700 Sequence Detection System, Applied Biosystem, December 1997) considering the level of GAPDH mRNA constant. All calculations of HCV RNA are expressed as fold difference over a specific control.

### Beta-Lactamase Gene Reporter Assay (BLA-Assay)

The BLA-assay was performed after 4 days of incubation in the presence of the compounds to be tested according to Murray et al., J Virol 77, 2928-35, 2003. Briefly, medium was removed, and cells were stained for 90 min with CCF4-AM (Aurora Biosciences Corp.) in Dulbecco's modified Eagle's medium supplemented with 25 mM HEPES, pH 8.0. For quantitation of the fraction of cells harboring bla replicons, cells were photographed by using a digital charge-coupled device color camera and green and blue cells were counted. Another method for measuring beta-lactamase activity is using a fluorescence plate reader that quantitates the amount of green (530 nm) or blue (460 nm) fluorescence emitted by cells stimulated with light of 405 nm.

### Cell ELISA Assays

The effect of the compounds of invention on viral replication and the replication proficiency of the mutant replicons was estimated by monitoring expression of the NS3 protein by Cell-ELISA with the anti-NS3 mab 10E5/24 as described by Trozzi et al. J. Virol. 2003, 77:3669-79). Compounds were dissolved and serially diluted in dimethyl sulfoxide (DMSO) in such a way that the final DMSO concentration was 1%. Transient transfection assays were performed with 10AIFN cells, prepared and transfected by electroporation as described by Trozzi et al. J. Virol. 2003, 77:3669-79). Cells were supplemented with the compounds between 1 and 4 hours after transfection

### Example 2

### Compound Synthesis

### Compound SB203580 was purchased from Calbiochem (San Diego, Calif. 92121). Compounds (1), (2) and (3) were obtained as described above.

### Example 3

### Inhibition of NS5A Phosphorylation in Cell Culture by Compounds of the Invention

Compounds of the present invention were evaluated in cell culture in order to assess their effect on NS5A phosphorylation in the context of live cells and active HCV replication using HB68 cells, which stably carry an adapted HCV replicon.

In order to follow NS5A hyperphosphorylation, cells were metabolically labeled with ³⁵S-methionine, or with ³²P-orthophosphate to investigate phosphorylation efficiency. Compounds (1) and (2) inhibited the formation of the hyperphosphorylated form of NS5A (p58; FIG. 1, lanes 3-4, 8-9 when used at a concentration of 5 μM. No compound inhibited basal NS5A phosphorylation without affecting NS5A expression. SB203580 was used as a negative control as it had no effect either on NS5A expression or on NS5A phosphorylation in cells at a concentration of 5 μM.

### Example 4

### Activation of Replication of wt Con1 Replicon in the Presence of Compounds of Invention—Detection by the bla-Gene Reporter Assay

To determine the biological consequences of inhibition of NS5A hyperphosphorylation, the effect of compounds of the present invention on HCV RNA replication effect was assessed in cell culture.

A subgenomic replicon was used in which the original neomycin phosphotransferase (neo) gene was replaced by the β-lactamase (bla) gene (WO2003089672 A1 and Murray et al., J Virol 77, 2928-2935, 2003). Cells actively replicating HCV express β-lactamase and show a fluorescent blue staining after incubation with a diffusible β-lactamase substrate (BLA-assay). Replicon RNA was electroporated in 10A-IFN cells and compounds were added at a concentration of 8 μM two hours after electroporation. The BLA-assay was performed after 4 days of incubation in the presence of the compounds to be tested according to Murray et al., J Virol 77, 2928-35, 2003. Briefly, medium was removed, and cells were stained for 90 min with CCF4-AM (Aurora Biosciences Corp.) in Dulbecco's modified Eagle's medium supplemented with 25 mM HEPES, pH 8.0. For quantitation of the fraction of cells harboring bla replicons, cells were photographed by using a digital charge-coupled device color camera and green and blue fluorescent cells were counted. Alternatively, fluorescence was measured by using a CytoFluor 4000 fluorescence plate reader.

Electroporation of the wild type Con1 replicon did not generate any fluorescent blue cells, whereas the addition of compound (1) or (2) used at a final concentration of 8 μM resulted in the production of fluorescent blue cells as a consequence of HCV replication. The control compound SB203580 had no effect on HCV replication. In order to demonstrate that the blue staining is a result of HCV replication and not a result of a longer half life of the electroporated HCV RNA or β-lactamase enzyme, the cells were incubated, in addition to the compounds, with an inhibitor of the HCV RNA-dependent RNA polymerase (Tomei et al., J. Virol. 78, 938-946, 2004). In the presence of a cell-permeable inhibitor of the HCV RNA-dependent RNA polymerase the number of observable fluorescent blue cells is significantly reduced.

### Example 5

### Activation of Replication of wt Con1 Replicon in the Presence of Compounds of Invention—Detection of HCV-RNA and HCV-Specific Proteins

It was investigated whether the compounds of the present invention activated HCV replication to an extent sufficiently efficient to allow the detection of viral proteins or viral RNA in the total cell population. RNA of wt Con1 replicon was electroporated into 10A-IFN cells and compounds were added two hours after electroporation. After 4 days of incubation, cells were collected and cellular extracts were assayed for the presence of NS5A by immunoblot (FIG. 2B) or for HCV RNA by quantitative PCR (FIG. 2A). As expected, no NS5A was visible in untreated cells or in cells incubated with the control inhibitor SB203580 at a concentration of 8 μM (FIG. 2B, lane 13 and 16). NS5A could be detected only in the presence of compound (1) (8 μM), whereas no protein was visible in cells treated with compound (2) (8 μM) (compare lane 14 with 15). Even though compound (2) induced replication, the efficiency was not high enough to be detectable by Western Blot. During the characterization of the replicon, several mutations were identified that were synergistic to adaptive mutations, thus increasing replication efficiency (Krieger et al., J. Virol 75, 4614-24, 2001; Trozzi et al., J Virol. 77, 3669-79, 2003). One of these mutations was E1202G, which maps in NS3 (herein described as m17). By itself this mutation had little if any effect in promoting replication of the Con1 replicon (FIG. 2B, lanes 2 and 5). However, it had a strong synergistic effect on replication when combined with the S2204A mutation (FIG. 2B, compare lanes 3 and 4). It was thus investigated whether the mutation E1202G in NS3 acted synergistically with the kinase inhibitors in a similar way to that observed with the adaptive mutation (lanes 5-8). Both compounds, used at a final concentration of 8 μM, activated replication of the replicon m17, with compound (1) as the more potent and compound (2) as the less potent activator. The synergistic effect of the mutation in NS3 with the adaptive mutation in NS5A (lanes 3 and 4) was comparable with its synergistic effect with the kinase inhibitors (compare lanes 3 and 4 with 14 and 6). The presence of NS5A was due to active HCV replication and not due to protein stabilization, because replicons containing the RdRP-inactivating mutation GAA (Lohmann et al., J. Virol. 71, 8416-28, 1997) did not show any detectable NS5A protein (lanes 9-12).

A similar experiment was carried out in order to detect HCV-specific RNA using real-time reverse transcription PCR amplification (FIG. 2A). Expression of β-actin was used as internal control in order to standardize for total amount of RNA. Shown is the fold-induction of HCV-RNA with respect to the DMSO control. Compound (1) induced replication of wt Con1 replicon 6-fold as measured by quantitative PCR, whereas induction of replication by the other compounds was below background (wt). As observed in FIG. 2B for protein expression, cells supporting subgenomic replicons with the synergistic mutation in NS3 (m17) contained significantly more HCV RNA upon incubation with the kinase inhibitors than those expressing the wt Con1 replicon. Replication was induced 312-fold for compound (1) and 58-fold for compound (2). The presence of HCV RNA was due to active replication as the HCV RNA polymerase-minus mutants (wt-GAA and m17-GAA) did not show any induced amount of RNA.

### Example 6

### Inhibitory Effect of the Compounds of the Invention on HCV Replicons

While the compounds of the present invention had a stimulatory effect on replicons that replicated inefficiently, they inhibited replicons that were fully competent for replication. The inhibitory effect of the compounds on the replication of HCV replicons was estimated by using cell lines transiently or stably transfected with HCV replicons. HCV replication and the effect of compounds were measured by using several different methods including Cell-ELISA, beta-lactamase and real-time reverse transcription PCR amplification (TaqMan) as described in Example 1. The inhibitory effect of the compounds on replication of HCV replicons depended on the expression level of hyperphosphorylated NS5A. Replicons expressing higher levels of the hyperphosphorylated form of NS5A were generally found to be less sensitive to inhibition by the compounds of the present invention, with IC₅₀ values ranging from 0.1 μM to about 10 μM. As an example, Con1 replicons carrying the adaptive mutation A2199T (AT) or S2204I (SI) were electroporated into 10A-IFN cells and increasing concentrations of compounds (1) or (2) were added two hours after electroporation. After 4 days of incubation, Cell-ELISA was performed. Both cell lines showed a dose-dependent inhibition of HCV replication with an IC₅₀ value of 0.5 μM for the SI replicon, which expresses low amounts of hyperphosphorylated NS5A and an IC₅₀ value of 5 μM for the AT replicon, which expresses high amounts of hyperphosphorylated NS5A.

### Example 7

### Compound Immobilization, Affinity Chromatography and Kinase Identification

Immobilization of compounds (1) and (3) was performed according to Godl et al., Proc. Natl. Acad. Sci. 26, 15434-15439, 2003. The experimental procedure is described briefly below.

For immobilization, drained epoxy-activated Sepharose 6B was resuspended in 2 vol of 20 mM a solution of either compound (1) or compound (3) in 50% dimethylformamide (DMF)/0.05 M Na₂CO₃. Coupling was performed overnight at 37° C. in the dark. After three washes with 50% DMF/0.05 M Na₂CO₃, remaining reactive groups were blocked with 1 M ethanolamine. Subsequent washing steps were performed according to the manufacturer's instructions. To generate the control matrix, epoxy-activated Sepharose 6B was incubated with 1 M ethanolamine and equally treated as described above. The beads were stored at 4° C. in the dark.

Frozen HuH-7 cells (2.5×10⁹) were lysed in 30 ml of buffer containing 20 mM Hepes (pH 7.5), 150 mM NaCl, 0.25% Triton X-100, 1 mM EDTA, 1 mM EGTA, 1 mM DTT plus additives (10 mM sodium fluoride/1 mM orthovanadate/10 μg/ml aprotinin/10 μg/ml leupeptin/1 mM phenylmethylsulfonylfluoride/10% glycerol), cleared by centrifugation, and adjusted to 1 M NaCl. The filtrated lysate was loaded with a flow rate of 100 μl/min on an HR 5/2 chromatography column (Amersham Biosciences) containing 600 μl of either compound (1) or compound (3) matrix equilibrated to lysis buffer without additives containing 1 M NaCl. The column was washed with 15 column volumes and equilibrated to lysis buffer without additives containing 20 mM NaCl, and bound proteins are eluted in the same buffer containing 1 mM compound (1) or compound (3) and 100 mM ATP with a flow rate of 100 μl/min. The volume of protein-containing elution fractions was reduced to 1/10 in a Centrivap concentrator (Labonco, Kansas City, Mo.) before precipitation according to Wessel and Flügge, Anal. Biochemistry 138, 141-143, 1984. Precipitated proteins were dissolved in 16-benzyldimethyl-n-hexadecylammonium chloride (16-BAC) sample buffer and after reduction/alkylation separated by two-dimensional gel electrophoresis. Silver-stained spots were picked and washed twice in 0.1 M ammonium bicarbonate (NH₄HCO₃) and reduced with 10 mMDTT in 0.1 M NH₄HCO₃ for 30 min at 56° C. Samples were then dehydrated with acetonitrile, rehydrated and alkylated with 55 mM iodoacetamide in 0.1 M NH₄HCO₃ for 30 min in the dark, and washed twice with 0.1 M NH₄HCO₃. Dried samples were reswollen in trypsin (Promega) solution containing 50 mM NH₄HCO₃ and digested overnight at 37° C. Peptides were washed out once with 50 mM NH₄HCO₃ and twice with 20% formic acid. Guanidination for matrix-assisted laser desorption ionization (MALDI) mass mapping was performed as described in Beardsley and Reilly, Anal. Chem. 74, 1884-1890, 2002. Sample cleanup was performed with ZipTips by using the manufacturer's procedures (Millipore). MALDI spectra were acquired by using a Bruker (Billerica, Mass.) Ultraflex time-of-flight (TOF)/TOF mass spectrometer with LIFT technology and anchor chip targets. Data analysis was performed by using Bruker's Biotools and the MASCOT program.

1. (canceled)

2. (canceled)

3. A method for modulating the replication of HCV RNA and/or viral production of HCV in a cell, a tissue or an organism comprising administering to the cell, the tissue or the organism an agent which inhibits the formation of hyperphosphorylated NS5A.

4. A method as claimed in claim 3 wherein the agent is:
N-methyl-4-{2-piperidin-4-yl-4-[3-(trifluoromethyl)phenyl]-1H-imidazol-5-yl}pyrimid-2-amine (1),
4-[5-(4-fluorophenyl)-2-(1-methylpiperidin-4-yl)-1H-imidazol-4-yl]pyridine (2), or
4-[5-(4-fluorophenyl)-4-pyridin-4-yl-1H-imidazol-2-yl]piperidine (3),
or a suitable salt thereof.

5. (canceled)

6. A method of enhancing HCV RNA replication and/or viral production of HCV in a cultured cell which comprises treating the cell with compound (1), (2) or (3) as defined in claim 4 or a suitable salt thereof.

7. A cell culture obtained by treating the cell with compound (1), (2) or (3) as defined in claim 4 or a suitable salt thereof.

8. A method of screening a compound for its effect on HCV replication which comprises administration of the compound to a HCV cell culture that has been treated with compound (1), (2) or (3) as defined in claim 4 or a suitable salt thereof.

9. (canceled)

10. A method of producing a cell culture which has detectable levels of HCV RNA in the absence of adaptive mutations in the HCV RNA by:
a) contacting a cell in tissue culture with HCV RNA or HCV virus not carrying adaptive mutations,
b) treating the cell with compound (1), (2) or (3) as defined in claim 4 or a suitable salt thereof, and
c) evaluating the treated cell for HCV RNA replication.

11. A method for producing a cell culture which has detectable levels of HCV protein in the absence of adaptive mutations in the HCV RNA by:
a) contacting a cell in tissue culture with HCV RNA or HCV virus not carrying adaptive mutations,
b) treating the cell with compound (1), (2) or (3) as defined in claim 4 or a suitable salt thereof, and
c) evaluating the treated cell for HCV protein expression.

12. A method of producing a cell culture which has detectable levels of virus production in the absence of adaptive mutations by:
a) contacting a cell in tissue culture with HCV RNA or HCV virus not carrying adaptive mutations,
b) treating the cell with compound (1), (2) or (3) as defined in claim 4 or a suitable salt thereof, and
c) evaluating the amount of viral particles secreted in the cell medium.

13. (canceled)

14. A method of producing a cell culture which has detectable levels of HCV RNA in the presence of selected adaptive mutations in those cells by:
a) contacting a cell in tissue culture with HCV RNA or HCV virus carrying selected adaptive mutations,
b) treating the cell with compound (1), (2) or (3) as defined in claim 4 or a suitable salt thereof, and
c) evaluating the treated cell for HCV RNA replication.

15. A method of producing a cell culture which has detectable levels of HCV protein in the presence of selected adaptive mutations in those cells by:
a) contacting a cell in tissue culture with HCV RNA or HCV virus carrying adaptive mutations,
b) treating the cell with compound (1), (2) or (3) as defined in claim 4 or a suitable salt thereof, and
c) evaluating the treated cell for HCV protein expression.

16. A method of producing a cell culture which has detectable levels of virus production in the presence of adaptive mutations by:
a) contacting a cell in tissue culture with HCV RNA or HCV virus carrying adaptive mutations,
b) treating the cell with compound (1), (2) or (3) as defined in claim 4 or a suitable salt thereof, and
c) evaluating the amount of viral particles secreted in the cell medium.

17. (canceled)

18. A method of identifying cellular kinase(s) responsible for the hyperphosphorylation of HCV NS5A by:
a) covalently binding compound (1), (2) or (3) as defined in claim 4 or a suitable salt thereof to a chromatography matrix,
b) using the chromatography matrix to purify kinase(s) from cellular protein extracts,
c) eluting the kinase(s) from the affinity matrix, and
d) identifying the eluted kinase(s).

19. (canceled)

20. A method of inhibiting replication of HCV RNA and/or of treating or preventing an illness due to hepatitis C virus, the method involving administering to a human or animal subject suffering from the condition a therapeutically or prophylactically effective amount of compound (1), (2) or (3) as defined in claim 4, or a pharmaceutically acceptable salt thereof.
